(19) United States
(12) Patent Application Publication
(10) Pub. No.: US 2026/0240541 A1
(43) Pub. Date: Aug. 20, 2026

(54) END TOOL AND SURGICAL INSTRUMENT INCLUDING THE SAME

(71) Applicant: LIVSMED INC., Seongnam-si (KR)

(72) Inventor: Woo Jung CHOI, Siheung-si (KR)

(21) Appl. No.: 19/459,612

(22) Filed: Jan. 26, 2026

(30) Foreign Application Priority Data
Feb. 18, 2025 (KR) 10-2025-0021175

(51) Int. Cl. A61B 17/072; A61B 17/00

(52) U.S. Cl. CPC A61B 17/07207; A61B 2017/00477; A61B 2017/07278

(57) ABSTRACT

An end tool of a surgical instrument includes a jaw configured to accommodate at least one region of an operation member configured to be movable in at least one direction, and at least one wire, including a first wire and a second wire, which are both connected to the operation member to allow the operation member to move in the at least one direction, where the first wire and the second wire are different wires.

### CROSS REFERENCE TO RELATED APPLICATION

This application is based on and claims priority under 35 USC §119 to Korean Patent Application No. 10-2025-0021175, filed on Feb. 18, 2025, in the Korean Intellectual Property Office, the disclosure of which is incorporated by reference herein in its entirety.

### BACKGROUND

### Field

The present disclosure relates to an end tool and a surgical instrument including the same.

### Description of the Related Art

Medically, surgery refers to the treatment of diseases by cutting, slitting, or manipulating the skin, mucous membranes, or other tissues using medical devices. In particular, open surgery, which cuts and opens the skin of a surgical site and cures, shapes, or removes an organ therein, may cause bleeding, side effects, patient pain, scars, or the like. Accordingly, recently, surgery performed by inserting only a medical device, for example, laparoscopic surgical instrument, microsurgical microscope, and the like by forming a predetermined hole in the skin or surgery using a robot has been spotlighted as an alternative.

A surgical instrument is a tool equipped with an end tool provided on one end of a shaft that passes through a hole drilled in the skin, and is manipulated by a medical doctor by hand using a predetermined driving part or by a robot arm to perform surgery at the surgical site. The end tool provided on the surgical instrument performs a rotational motion, a gripping motion, a cutting motion, or the like through a predetermined structure.

The background art described above is technical information retained by the present inventors in order to derive the present disclosure or obtained by the present inventors in the process of deriving the present disclosure, and thus is not necessarily known art disclosed to the general public before the filing the application of the present disclosure.

### SUMMARY

The present disclosure is directed to providing an end tool and a surgical instrument including the same, capable of utilizing a combination of different cables during laparoscopic surgery or various other surgical procedures.

According to an aspect of the present disclosure, an end tool of a surgical instrument may include a jaw configured to accommodate at least one region of an operation member configured to be movable in at least one direction, and at least one wire including a first wire and a second wire, which are both connected to the operation member to allow the operation member to move in the at least one direction, wherein the first wire and the second wire are different wires.

In another embodiment of the present disclosure, the first wire may be an advancing wire configured to transmit a driving force to cause the operation member to move forward toward a distal end of the jaw, and the second wire may be a retraction wire configured to transmit a driving force to cause the operation member to move backward toward a proximal end of the jaw.

In the other embodiment of the present disclosure, materials included in the first wire and the second wire may be different.

In the other embodiment of the present disclosure, the first wire may include a metal material, and the second wire may include a polymer.

In the other embodiment of the present disclosure, one of the first wire and the second wire may include stainless steel, and a remaining wire of the first wire and the second wire may include tungsten.

In the other embodiment of the present disclosure, the first wire may include tungsten having a purity of 95% or more, and the second wire may include stainless steel having an iron content of less than 80%.

In the other embodiment of the present disclosure, the first wire may have a greater diameter than the second wire.

In the other embodiment of the present disclosure, the first wire may have a greater tensile strength than the second wire.

In the other embodiment of the present disclosure, the end tool may further include at least one pulley having a preset central axis of rotation and configured to be in contact with the at least one wire, wherein the first wire and the second wire may be wound around the at least one pulley to control at least one degree of freedom of the jaw.

In the other embodiment of the present disclosure, when the at least one pulley rotates in one direction, the first wire and the second wire may move in different directions.

According to another aspect of the present disclosure, an end tool of a surgical instrument may include a jaw configured to accommodate at least one region of an operation member configured to be movable in at least one direction, a first wire connected to the operation member to allow the operation member to move in the at least one direction, and a third wire configured to transmit a driving force to cause the jaw to perform a rotational motion around a first shaft, wherein the first wire and the third wire are different wires.

In the other embodiment of the present disclosure, the first wire may be an advancing wire configured to transmit a driving force to cause the operation member to move forward toward a distal end of the jaw, and the third wire may be a yaw wire corresponding to a yaw motion of the end tool.

In the other embodiment of the present disclosure, the first wire may be an advancing wire configured to transmit a driving force to cause the operation member to move forward toward a distal end of the jaw, and the third wire may be a pitch wire corresponding to a pitch motion of the end tool.

In the other embodiment of the present disclosure, materials included in the first wire and the third wire may be different.

In the other embodiment of the present disclosure, the first wire may include a metal material, and the third wire may include a polymer.

In the other embodiment of the present disclosure, the first wire may include tungsten having a purity of 95% or more, and the third wire may include stainless steel having an iron content of less than 80%.

In the other embodiment of the present disclosure, the first wire may have a greater diameter than the third wire.

In the other embodiment of the present disclosure, the first wire may have a greater tensile strength than the third wire.

According to the other aspect of the present disclosure, a surgical instrument may include the end tool according to an embodiment of the present disclosure or according to another embodiment of the present disclosure, a manipulation part configured to control a motion of the end tool, and a connection part configured to connect the manipulation part to the end tool.

In the other embodiment of the present disclosure, the end tool may be configured to perform a yaw rotation around a first shaft and a pitch rotation around a second shaft different from the first shaft.

According to the other aspect of the present disclosure, a pulley fixing pin applicable to a surgical instrument may include a body portion extending in a longitudinal direction and configured to pass through a fixed pulley around which a wire is wound, and a flange portion positioned on one side of the body portion, wherein a first diameter of an outermost circumferential surface of the flange portion is greater than a second diameter of an outer circumferential surface of the body portion.

In the other embodiment of the present disclosure, the first diameter of an outer circumferential surface of the flange portion may increase in a direction away from the body portion.

In the other embodiment of the present disclosure, a groove of a preset depth may be defined on an inner side of the flange portion.

Other aspects, features, and advantages other than those described above will become apparent from the following drawings, claims, and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings.

FIG. 1 is a perspective view illustrating a surgical instrument to which an end tool according to an embodiment of the present disclosure is applied.

FIG. 2 is a perspective view illustrating a reload assembly to which the end tool according to an embodiment of the present disclosure is applied.

FIG. 3 is a perspective view illustrating the end tool according to an embodiment of the present disclosure.

FIG. 4 is a perspective view of the end tool of FIG. 3 with a cartridge removed, viewed from another direction.

FIG. 5 is a schematic perspective view illustrating an operation member, a wedge member, fixed pulleys, and a wire unit of the end tool of FIG. 3.

FIG. 6 is a cross-sectional view taken along line I-I’ of FIG. 3.

FIG. 7 is a perspective view of a pulley fixing pin of FIG. 6.

FIGS. 8 and 9 are schematic views illustrating forward and backward movements of the operation member of the end tool of FIG. 5.

FIG. 10 is an exploded perspective view of the end tool of FIG. 3.

FIG. 11 is a perspective view illustrating the end tool according to an embodiment of the present disclosure.

FIG. 12 is a perspective view illustrating an end tool hub and a pitch hub of the end tool of FIG. 11.

FIG. 13 is a perspective view illustrating a rotational shaft and pulley structure of the end tool of FIG. 11.

FIG. 14 is a perspective view illustrating a connection relationship among pulleys, the operation member, and wire units.

FIG. 15 is a side view of the end tool of FIG. 11 with the end tool hub removed.

FIG. 16 is a perspective view of the end tool of FIG. 11 with the end tool hub removed.

FIG. 17 is a perspective view of the end tool of FIG. 16 viewed from another direction.

### DETAILED DESCRIPTION

Hereinafter, the following embodiments will be described in detail with reference to the accompanying drawings. When describing with reference to the drawings, identical or corresponding components will be assigned the same reference numerals and duplicate descriptions thereof will be omitted.

Since various transformations can be made to these embodiments, specific embodiments will be illustrated in the drawings and described in detail in the detailed description. The effects and features of the present embodiments and the accompanying methods thereof will become apparent from the following description of the contents, taken in conjunction with the accompanying drawings. However, the present embodiments are not limited to the embodiments disclosed below, but may be implemented in various forms.

In describing the present disclosure, a detailed description of known related arts will be omitted when it is determined that the gist of the present disclosure may be unnecessarily obscured.

In the following embodiments, singular forms are intended to include plural forms as well, unless the context clearly indicates otherwise. Although terms such as “first,” “second,” and the like may be used to describe various components, such components should not be limited to the above terms The terms are only used to distinguish one component from another.

In the following embodiments, terms such as “include” or “have” means that the features or components described in the specification are present, and the possibility that one or more other features or components will be added is not excluded in advance.

In the following embodiments, when a unit, region, or component is referred to as being formed on another unit, region, or component, it can be directly formed on the other unit, region, or component. That is, for example, intervening units, regions, or components may be present.

In the following embodiments, terms such as “connecting” or “coupling” two members do not necessarily mean a direct and/or fixed connection or coupling of the two members, unless the context clearly indicates otherwise, and do not preclude another members from being interposed between the two members.

Sizes of components in the drawings may be exaggerated or reduced for convenience of description. For example, since the size and thickness of each component shown in the drawings are arbitrarily illustrated for convenience of description, the following embodiments are not necessarily limited thereto.

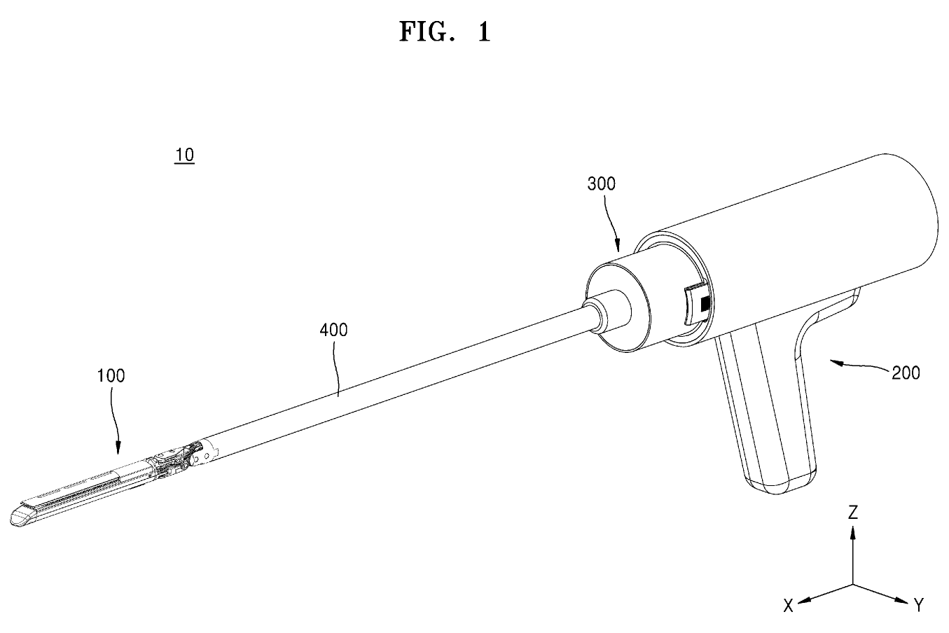

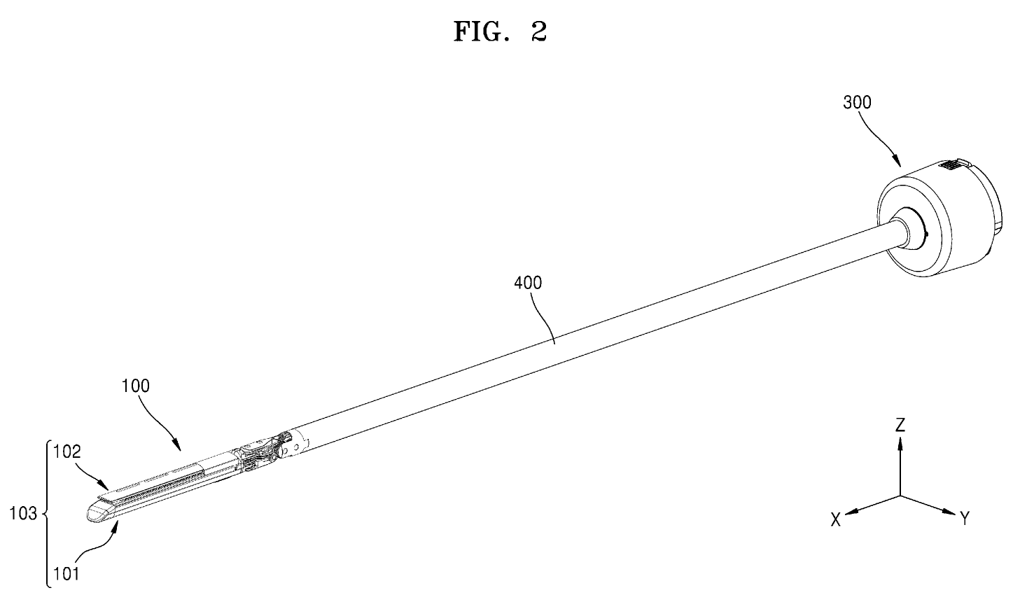

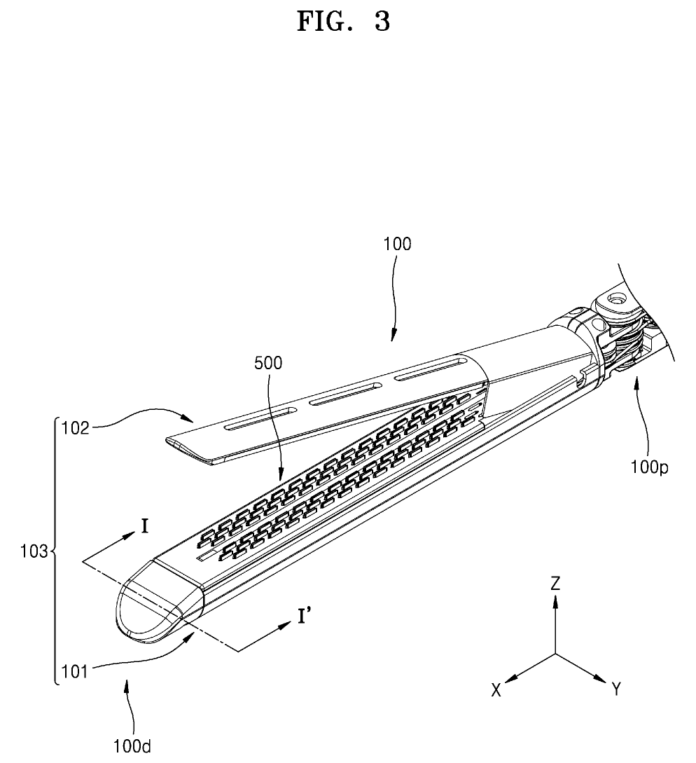

FIG. 1 is a perspective view illustrating a surgical instrument to which an end tool according to an embodiment of the present disclosure is applied, and FIG. 2 is a perspective view illustrating a reload assembly to which the end tool according to an embodiment of the present disclosure is applied. FIG. 3 is a perspective view illustrating the end tool according to an embodiment of the present disclosure.

In describing the present disclosure, the portion closer to a user side, i.e., the portion closer to a manipulation part 200, will be referred to as a proximal end, and the portion farther from the user side, that is, the portion closer to an end portion of an end tool 100, will be referred to as a distal end.

For example, the portion of the end tool 100 closer to a connection part (or shaft) 400 will be defined as a proximal end 100p, and the portion farther from the connection part (or shaft) 400, that is, the part closer to the end portion of the end tool 100 will be defined and described as a distal end 100d.

The end tool 100 according to an embodiment of the present disclosure may be connected to one end portion of the connection part (shaft) of a surgical instrument and inserted into a surgical site to perform motions required for surgery.

First, the surgical instrument and the reload assembly, to which the end tool 100 according to an embodiment of the present disclosure is applicable, will be described.

Referring to FIGS. 1 to 3, the surgical instrument according to the present embodiment may include the end tool 100, the manipulation part 200, the connection part 400, and a power transmission part 300.

In some embodiments, as shown in FIG. 2, a configuration including the end tool 100, the connection part (or shaft) 400, and the power transmission part 300 will be distinguished and referred to as the reload assembly.

Here, the connection part 400 is formed in the shape of a hollow shaft, and one or more wires and electric wires may be accommodated therein. For convenience of description of the present embodiment, the shaft will be referred to and described as the connection part 400. The manipulation part 200 is coupled to one end portion of the connection part 400, the end tool 100 is coupled to another end portion thereof, and the connection part 400 may serve to connect the manipulation part 200 to the end tool 100. As an example, the connection part 400 may include a straight part, and although not shown in the drawings, the connection part 400 may include one or more curved parts to increase ease of use and control the arrangement of components for manipulation.

The power transmission part 300 may be formed on one end portion of the connection part 400 and may serve to transmit power generated from a power generation part to be described later to the end tool 100. For example, the power transmission part 300 may be positioned between the end tool 100 and the manipulation part 200. As will be described later, when a user such as a medical doctor manipulates the manipulation part 200, the power generation part generates power to control the end tool 100, and the generated power may be transmitted to the end tool 100 through the power transmission part 300. The power transmission part 300 may include a plurality of wires, a plurality of pulleys, a plurality of links, a plurality of joints, a plurality of gears, and the like.

A user may operate the end tool 100 by manipulating the manipulation part 200. For example, the manipulation part 200 is a configuration for the user to input signals to control the motions of the end tool 100. That is, the manipulation part 200 may be said to be a configuration that receives signals from the user to control the operation of the end tool 100. Here, the signals for controlling the motions of the end tool 100 may correspond to mechanical manipulations such as pressing a button or switch, or rotating or moving a particular member, and may also be electrical signals generated by such mechanical manipulations, but the present disclosure is not limited thereto.

The manipulation part 200 is provided as an interface to be directly controlled by a medical doctor, for example, provided in a gun shape, a tongs shape, a stick shape, a lever shape, or the like, and when the medical doctor controls the manipulation part 200, the end tool 100, which is connected to the corresponding interface and inserted into the body of a surgical patient, performs a certain motion, thereby performing surgery. Here, the manipulation part 200 is illustrated in FIG. 1 as being formed in a gun shape, but the concept of the present disclosure is not limited thereto, and various types of manipulation parts that can be connected to the end tool 100 and manipulate the end tool 100 may be possible.

The manipulation part 200 may include a housing forming the exterior of the manipulation part 200. At least a portion of the power generation part configured to generate power to control the end tool 100 may be accommodated inside the housing. Further, a circuit unit for controlling the operation of the power generation part and a slip ring for supplying electrical energy to the power generation part, connecting communication, or transmitting various other signals may be accommodated inside the housing.

A handle may be formed on the manipulation part 200. The handle is a part for a user to grip. Thus, the user can use a surgical instrument 10 according to the present disclosure while gripping the handle of the manipulation part 200.

For example, although not shown in the drawings, a button, a switch, a lever, and the like for controlling various motions of the end tool 100 may be further formed in the manipulation part.

The end tool 100 is formed on another end portion of the connection part 400, and performs necessary motions for surgery by being inserted into a surgical site. As an example of the end tool 100, a pair of jaws 103 for performing a grip motion may be used. However, the concept of the present disclosure is not limited thereto, and various devices for performing surgery may be used as the end tool 100. For example, a configuration such as a cantilever cautery may also be used as the end tool. The end tool 100 is connected to the manipulation part 200 by the power transmission part 300 (e.g., a wire or the like), and receives a driving force of the manipulation part 200 through the power transmission part 300 to perform a motion necessary for surgery, such as gripping, cutting, suturing, or the like.

Hereinafter, the end tool 100 of the surgical instrument of FIG. 3 will be described in more detail.

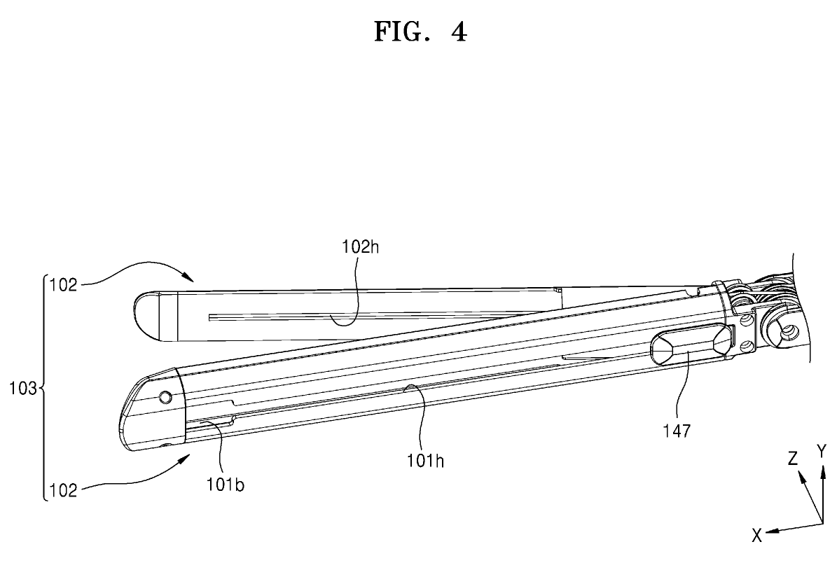

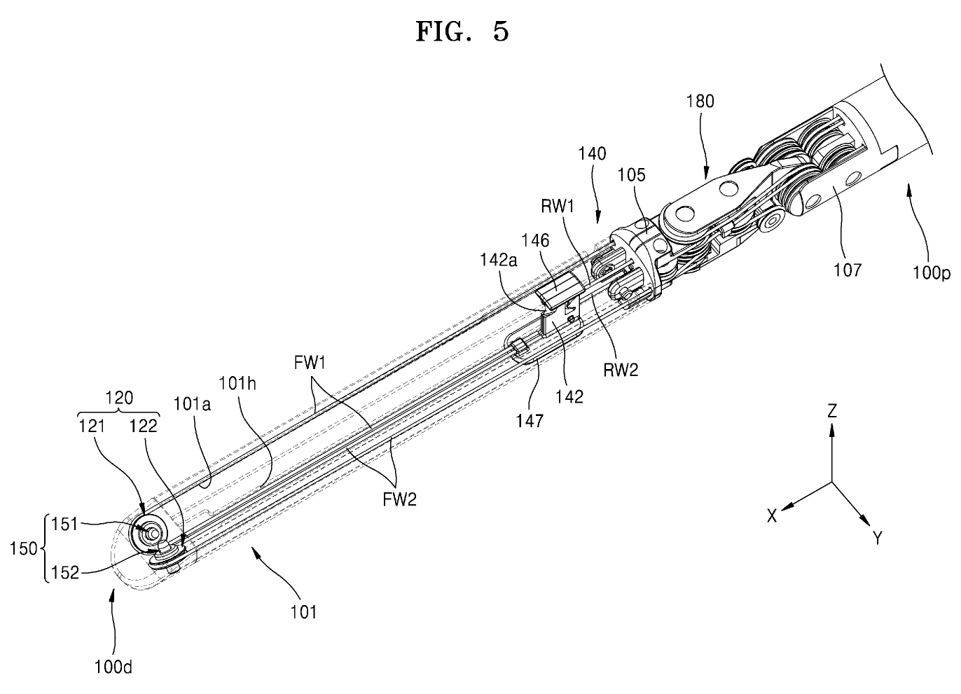

FIG. 4 is a perspective view of the end tool of FIG. 3, viewed from another direction, with the cartridge removed, and FIG. 5 is a schematic perspective view for describing an operation member, a wedge member, fixed pulleys, and a wire unit of the end tool of FIG. 3.

The end tool 100 may include the jaw 103, a plurality of fixed pulleys 120, and wire units FW and RW, the wire unit having at least one wire. The plurality of fixed pulleys 120 may include two or more pulleys, for example, a first fixed pulley 121 and a second fixed pulley 122. The wire units FW and RW include two or more wires, and may include, for example, a first wire FW and a second wire RW.

The first wire FW may be an advancing wire that transmits a driving force to move an operation member 140 forward toward the distal end 100d of the jaw 103. The first wire FW may include two or more wires, for example, a first advancing wire FW1 and a second advancing wire FW2.

The second wire RW may be a retraction wire that transmits a driving force to move the operation member 140 backward toward the proximal end of the jaw 103. The second wire RW may include two or more wires, for example, a first retraction wire RW1 and a second retraction wire RW2.

The jaw 103 may perform various functions, such as a grip motion, and may include a pair of jaws, e.g., a first jaw 101 and a second jaw 102 as a specific example. Here, each of the first jaw 101 and the second jaw 102, or a component encompassing the first jaw 101 and the second jaw 102 may be referred to as the jaw 103.

The first jaw 101 and the second jaw 102 may be positioned to face each other, may move closer to or move away from each other, and may be configured to rotationally move around, for example, a first shaft.

The cartridge 500 may be positioned to be accommodated in the first jaw 101, and a plurality of staples 530 are positioned inside the cartridge 500. When an operation member 140 receives a force through a plurality of advancing wires FW1 and FW2 while the first jaw 101 and the second jaw 102 are close to each other, such as when the first jaw 101 and the second jaw 102 are closed with body tissue interposed therebetween, the operation member 140 may push the staples 530 upward while moving toward a distal end 100d of the first jaw 101, thereby performing stapling.

At this point, one or more clamps 146 and 147 of the operation member 140 may protrude to the outside of the first jaw 101 and the second jaw 102, allowing the operation member 140 to move forward while applying a pressure to an outer surface of the first jaw 101 and the second jaw 102, which facilitates the smooth progression of a stapling process. In an optional embodiment, the cartridge 500 may have a housing 520 corresponding to the bottom thereof and may have a form in which the housing 520 is positioned in the first jaw 101.

The operation member 140 may include a body 142, a first clamp 146, and a second clamp 147. In some embodiments, the operation member 140 may be used together with a wedge 540. For example, the wedge 540 may be prepared separately from the operation member 140 and then disposed adjacent to the operation member 140 in the first jaw 101. In some embodiments, as another example, the operation member 140 and the wedge 540 may be integrally formed as one body. In the present specification, for convenience of description, the operation member 140 and the wedge 540 will be described and illustrated in the drawings with the assumption that the operation member 140 and the wedge 540 are prepared separately.

The wedge 540 may be positioned on at least one side of the body 142 and may be formed to have a predetermined inclined surface. That is, the wedge 540 may be formed to be inclined by a certain degree with respect to an extension direction of the end tool 100. For example, the wedge 540 may be formed to have a greater height at the proximal end 100p side of the first jaw 101 than the distal end 100d side of the first jaw 101.

The wedge 540 may be configured to be sequentially in contact with withdrawal members 535 or the plurality of staples 530 positioned in the cartridge 500, and may serve to sequentially push the staples 530 upward.

The plurality of fixed pulleys 120 may be disposed in the first jaw 101 to be closer to the front of the cartridge 500, i.e., to the distal end 100d of the first jaw 101, than the cartridge 500, and details thereof will be described later.

The operation member 140 of the end tool 100 will be described in more detail.

Referring to FIGS. 4 and 5, the body 142 may have an elongated columnar shape and, for example, may be in the form of a plate-shaped column. For example, a blade 142a may be formed in one region of the body 142, and an edge sharply formed to cut tissue may be formed in the blade 142a. The tissue positioned between the first jaw 101 and the second jaw 102 may be cut as at least a portion of the edge positioned in the blade 142a of the body 142 is withdrawn to the outside of the first jaw 101 and the cartridge 500.

The first clamp 146 may be formed in one region of the body 142, and the second clamp 147 may be formed in another region different from the one region. For example, the body 142 may be positioned between the first clamp 146 and the second clamp 147.

The first clamp 146 and the second clamp 147 may have a region with a width at least greater than that of the body 142. Accordingly, the first clamp 146 may be inserted into and pass through a guide groove 102a formed in the second jaw 102 in the longitudinal direction to be positioned or brought into contact with an upper surface of the second jaw 102 and, at the same time, the second clamp 147 may be inserted into and pass through a guide groove 101h formed in the first jaw 101 in the longitudinal direction to be positioned or brought into contact with a lower surface of the first jaw 101, so that the first clamp 146 and the second clamp 147 may move. Thus, when the operation member 140 moves, the first clamp 146 and the second clamp 147 may apply forces in directions that bring the second jaw 102 and the first jaw 101 closer to each other.

As a result, when the operation member 140 moves from the proximal end 100p of the first jaw 101 toward the distal end 100d of the first jaw 101, a motion of decreasing a distance between the second jaw 102 and the first jaw 101, i.e., a closing motion of the jaw 103, may be naturally implemented through the first clamp 146 and the second clamp 147.

The first clamp 146 and the second clamp 147 may be positioned at different locations relative to a forward-facing direction relative to the body 142. For example, the second clamp 147 may be positioned further forward than the first clamp 146, for example, the second clamp 147 may be positioned closer to the distal end 100d of the first jaw 101 than the first clamp 146 when the operation member 140 is positioned in the first jaw 101. Accordingly, the operation member 140 may move forward while the first jaw 101 and second jaw 102 are in the closed state, so that the first jaw 101 and second jaw 102 can be maintained with greater efficiency and stability while performing stapling.

The jaw 103 of the end tool 100 will be described in more detail.

Referring to FIGS. 3 to 5, the first jaw 101 is formed in an elongated bar shape as a whole, and a rotational shaft may be disposed in the proximal end 100p such that the first jaw 101 is rotationally movable, and such a rotational shaft may correspond to the rotational shaft formed in the second jaw 102 described above. For example, the cartridge 500 may be accommodated on a side closer to the distal end 100d than to the rotational shaft.

For example, the first jaw 101 may be formed entirely in the form of a hollow box with one surface (upper surface) thereof removed, such that a cartridge accommodation part 101a capable of accommodating the cartridge 500 is formed inside the first jaw 101. That is, the first jaw 101 may be formed in an approximately “U” shape in cross section.

The guide groove 101h may be formed in a bottom surface of the first jaw 101, the bottom surface opposite to an upper open region formed by removing one surface. In some embodiments, the guide groove 101h may be configured to guide a linear motion of the operation member 140.

Referring to FIG. 4, the guide groove 101h may be configured to guide the operation member 140, and may have a through-hole shape in a region facing the operation member 140. Through this, one region of the operation member 140, such as at least one region of the operation member 140, or the second clamp 147 connected thereto may pass through the guide groove 101h and be ejected to the outside of the first jaw 101. When the operation member 140 moves forward, the second clamp 147 may pass through the guide groove 101h of the first jaw 101 to be exposed to the outside of the first jaw 101, and may come into contact with the lower surface of the first jaw 101 or apply pressure thereto. As the operation member 140 moves, the second clamp 147 applies pressure on the lower surface of the first jaw 101 and the first clamp 146 applies pressure on the upper surface of the second jaw 102 such that a gap between the second jaw 102 and the first jaw 101 decreases, allowing the second jaw 102 to naturally remain in a closed state with respect to the first jaw 101.

In an optional embodiment, the first jaw 101 may include a window 101b. After operating the operation member 140 or using the end tool 100, the second clamp 147 of the operation member 140 may be positioned corresponding to the window 101b, and the coupled state of the first jaw 101 and the operation member 140 may be released.

The plurality of fixed pulleys 120 may be disposed in front of the cartridge accommodation part 101a of the first jaw 101, and, for example, the first fixed pulley 121 and the second fixed pulley 122 may be disposed.

The first fixed pulley 121 and the second fixed pulley 122 may be disposed to have an inclined shape rather than being disposed parallel to each other. For example, the first fixed pulley 121 and the second fixed pulley 122 may be disposed such that a distance therebetween decreases in a direction away from the second jaw 102.

In some embodiments, the first fixed pulley 121 and the second fixed pulley 122 may be disposed in the first jaw 101 to face each other, and, for example, the first fixed pulley 121 and the second fixed pulley 122 may be disposed symmetrically to each other. For example, the first fixed pulley 121 and the second fixed pulley 122 may have the same size.

The first advancing wire FW1 and the second advancing wire FW2 may be correspondingly wound around the first fixed pulley 121 and the second fixed pulley 122, respectively. One region of each of the first advancing wire FW1 and the second advancing wire FW2, which are wound around the first fixed pulley 121 and the second fixed pulley 122 to be drawn out from lower sides thereof, may be directed toward the operation member 140 described above.

Through such a configuration, the surgical instrument 10 according to an embodiment of the present disclosure may improve balance characteristics of the arrangement of the first and second fixed pulleys 121 and 122 and the first and second advancing wires FW1 and FW2 with respect to the moving direction of the operation member 140, thereby facilitating the implementation of, for example, a symmetrical configuration.

The surgical instrument 10 according to an embodiment of the present disclosure may reduce or prevent shaking of the end tool 100 by decreasing the occurrence of vibration or rotational moment when the first advancing wire FW1 and the second advancing wire FW2 are pulled.

The surgical instrument 10 according to an embodiment of the present disclosure may implement a compact overall structure of the end tool 100 by forming the end tool 100 such that a width of a main region on one side, specifically, a lower side, of the first jaw 101, which is one side of the jaw 103, is reduced.

In the surgical instrument 10 according to an embodiment of the present disclosure, the first fixed pulley 121 and the second fixed pulley 122 may be disposed in an inclined manner rather than being parallel to each other.

Accordingly, certain regions of the first advancing wire FW1 and the second advancing wire FW2 according to an embodiment of the present disclosure may be respectively wound around the first fixed pulley 121 and the second fixed pulley 122, drawn out from lower sides of the first fixed pulley 121 and the second fixed pulley 122, and connected to the operation member 140. Other regions of the first advancing wire FW1 and the second advancing wire FW2 may be respectively wound around the first fixed pulley 121 and the second fixed pulley 122, drawn out from upper sides of the first fixed pulley 121 and the second fixed pulley 122, and positioned on both sides of the cartridge 500, i.e., between the cartridge 500 and the respective side surfaces of the first jaw 101.

In the surgical instrument 10 according to an embodiment of the present disclosure, by positioning the first advancing wire FW1 and the second advancing wire FW2 to correspond to the first fixed pulley 121 and the second fixed pulley 122, which are inclined on both sides, for example, in a symmetrical configuration, the maximum tension applied to the first advancing wire FW1 and the second advancing wire FW2 may be the same or substantially similar, thereby improving the fatigue life of each wire.

In the surgical instrument 10 according to an embodiment of the present disclosure, the two advancing wires FW1 and FW2 may be controlled such that certain regions symmetrically pass below the cartridge 500 and other regions respectively pass along both sides of the cartridge 500. Accordingly, unwanted moments or rotational forces that may occur when the two advancing wires FW1 and FW2 are pulled, resulting in unintended movement or vibration of the end tool 100 or the surgical instrument 10 including the same, may be reduced or prevented.

A plurality of pulley fixing pins 150 configured to fix the plurality of fixed pulleys 120 to the jaw may be disposed at a front side of the first jaw. The plurality of fixed pulleys 120 may be axially coupled to the plurality of pulley fixing pins 150.

In some embodiments, the first fixed pulley 121 may be axially coupled to a first pulley fixing pin 151, and the second fixed pulley 122 may be axially coupled to a second pulley fixing pin 152.

The plurality of pulley fixing pins 150 may be fixed to the first jaw 101 to fix positions of the fixed pulleys 120, and may function as rotational shafts for allowing the plurality of fixed pulleys 120 to rotate.

When the first fixed pulley 121 and the second fixed pulley 122 are disposed in an inclined manner rather than being parallel to each other, the first pulley fixing pin 151 and the second pulley fixing pin 152 may also be disposed in an inclined manner with respect to each other. For example, the plurality of pulley fixing pins 150 may be disposed symmetrically to each other.

The arrangement relationship of the plurality of pulley fixing pins 150, the fixed pulleys 120 and the first jaw 101 will be described in more detail.

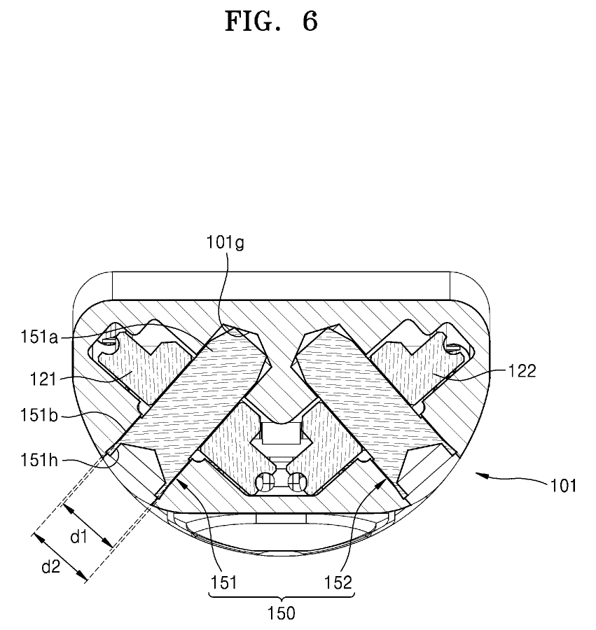

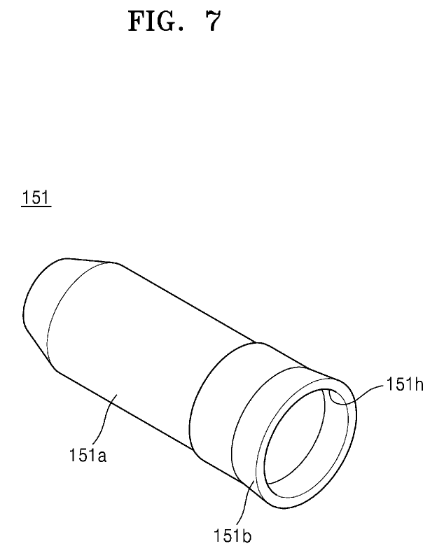

FIG. 6 is a cross-sectional view taken along line I-I’ of FIG. 3, and FIG. 7 is a perspective view of the pulley fixing pin of FIG. 6.

Referring to FIGS. 6 and 7, the plurality of pulley fixing pins 150 are disposed to pass through the plurality of fixed pulleys 120, respectively, to be fixed to the first jaw 101. The plurality of pulley fixing pins 150 may each be disposed to form a preset angle with a respective one of the plurality of fixed pulleys 120. For example, the plurality of pulley fixing pins 150 may each be disposed perpendicular to a respective one of the plurality of fixed pulleys 120.

The first pulley fixing pin 151 and the second pulley fixing pin 152 may be formed in the same shape. Hereinafter, the shape of the plurality of pulley fixing pins 150 will be described based on the first pulley fixing pin 151.

The first pulley fixing pin 151 extends in a longitudinal direction and may be inserted into a fixing hole 101g formed in the distal end of the first jaw 101. The fixing hole 101g may be formed as a blind hole with a preset depth, having an open end on one side and a closed end on another side opposite to the one side.

That is, when the first pulley fixing pin 151 is inserted into the fixing hole 101g, one side of the first pulley fixing pin 151 may be exposed to the outside of the first jaw 101, but another side opposite to the one side may be positioned at the closed end of the fixing hole 101g and may not be exposed to the outside.

One side of the first pulley fixing pin 151 may be positioned so as to be in contact with the closed end of the fixing hole 101g.

The first pulley fixing pin 151 may include a body portion 151a and a flange portion 151b.

The body portion 151a may extend in a longitudinal direction and may be configured to pass through the first fixed pulley around which a wire is wound, and a diameter of an outer circumferential surface of the body portion 151a may be smaller than a diameter of an inner circumferential surface of the fixing hole 101g.

An inclined surface that is inclined inward may be formed at one end portion of the body portion 151a. For example, the body portion 151a may gradually decrease in diameter toward the end portion thereof.

The one end portion of the body portion 151a, where the inclined surface is formed, may be a region that first begins to be inserted when the first pulley fixing pin 151 is inserted into the fixing hole 101g. Accordingly, the first pulley fixing pin 151 may be easily inserted into the fixing hole 101g.

The flange portion 151b may be positioned on one side of the body portion 151a and may have a groove with a preset depth formed therein, and a diameter of an outer circumferential surface of the flange portion 151b may increase in a direction away from the body portion 151a.

The flange portion 151b may be positioned to face the inclined surface of the body portion 151a. For example, an inclined surface may be formed at one end portion of the pulley fixing pin, and the flange portion 151b may be formed at another end portion of the pulley fixing pin opposite to the one end portion.

A flange groove 151h having a preset depth may be formed in the flange portion 151b. The depth of the flange groove 151h may increase from an outer edge of the first pulley fixing pin 151 toward a center thereof. An axial depth of the flange groove 151h may be relatively greater than a circumferential depth thereof.

The diameter of the outer circumferential surface of the flange portion 151b may increase in a direction away from the body portion 151a, i.e., toward one end portion of the first pulley fixing pin 151 exposed to the outside. For example, the outer circumferential surface of the flange portion 151b may be inclined with respect to the outer circumferential surface of the body portion 151a.

For example, the outer circumferential surface of the flange portion 151b may be formed in a shape in which a distance from a central axis gradually increases, and may be inclined with respect to an axial direction.

Accordingly, a diameter d1 of the outer circumferential surface of the flange portion 151b connected to the body portion 151a may be formed to be smaller than a diameter d2 of the outer circumferential surface of the flange portion 151b positioned at the outermost side of the first pulley fixing pin 151.

The diameter d1 of the outer circumferential surface of the flange portion 151b connected to the body portion 151a may be formed to be relatively smaller than the diameter of the inner circumferential surface of the fixing hole 101g, and the diameter d2 of the outer circumferential surface of the flange portion 151b positioned at the outermost side of the first pulley fixing pin 151 may be formed to be relatively larger than the diameter of the inner circumferential surface of the fixing hole 101g.

As described above, when the diameter d2 of the outer circumferential surface of the flange portion 151b positioned at the outermost side of the first pulley fixing pin 151 is relatively larger than the diameter of the inner circumferential surface of the fixing hole 101g, and a groove is formed inside the flange portion 151b, the flange portion 151b may be easily deformed inward and press-fitted into the fixing hole 101g by simply inserting the first pulley fixing pin 151 into the fixing hole 101g. Accordingly, the first pulley fixing pin 151 may be stably positioned and fixed in the fixing hole 101g through simple insertion, without requiring a separate tool.

In the related art, in order to fix a cylindrical component such as a rotational shaft to a surgical tool, methods such as caulking, in which an end portion of the component is inserted into a hole and then deformed, or welding have been used.

In the caulking method, the component is inserted through one side of the hole, and the portion of the component exposed to another side of the hole is deformed to have a diameter larger than that of the hole for achieving fixation. However, when using the caulking method, there was a problem in that the hole must pass through the surgical tool so that opposite end portions of the component are exposed to the outside.

In the welding method, since the component is completely fixed in the hole, there was a problem in that the component may become damaged because the component cannot properly absorb deformation that occurs during the fabrication and assembly of other components of the surgical tool. In some embodiments, there was a limitation in that the component and the end tool to which the component is fixed had to be made of materials that are weldable to each other.

In the pulley fixing pin 150 according to an embodiment of the present disclosure, the flange portion 151b having an inclined shape is deformed and fixed inside the fixing hole 101g by simply inserting the pulley fixing pin 150 into the fixing hole 101g, thereby providing an advantage in that no separate tool is required for fixation. This also allows the pulley fixing pin 150 to be easily removed without damaging other parts.

For example, since it is not necessary for both sides of the pulley fixing pin 150 to be exposed to the outside, the fixing hole 101g can be formed as a blind hole having a closed end on one side, and the position at which the pulley fixing pin 150 is fixed may be more freely adjusted, thereby providing an advantageous effect.

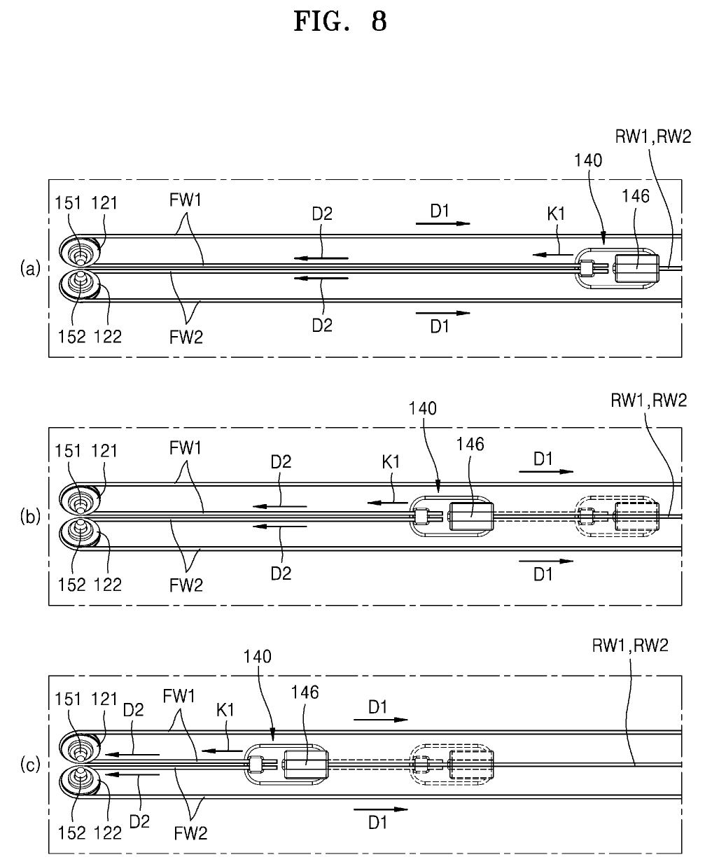

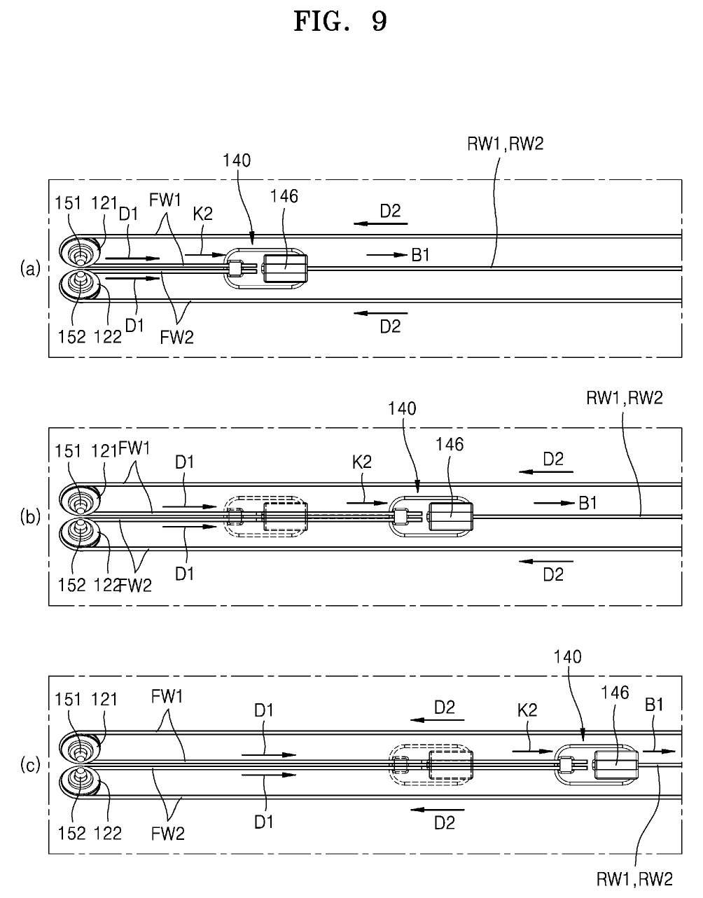

FIGS. 8 and 9 are schematic views for describing forward and backward movements of the operation member of the end tool of FIG. 5.

Referring to FIGS. 8 and 9, for convenience of description, the first jaw 101 is excluded, and the first advancing wire FW1, the second advancing wire FW2, the second wire RW, the first fixed pulley 121, the second fixed pulley 122, and the operation member 140 are illustrated.

Based on FIG. 8, the operation member 140 may move in a leftward direction, i.e., move forward toward the distal end 100d, and this forward movement is illustrated sequentially in (a), (b), and (c) of FIG. 8.

As shown in (a) of FIG. 8, when the first advancing wire FW1 and the second advancing wire FW2 are pulled in a first direction D1, one region of each of the first advancing wire FW1 and the second advancing wire FW2 is pulled in the first direction D1, and thus the regions of the first advancing wire FW1 and the second advancing wire FW2 emerging from the lower sides of the first fixed pulley 121 and the second fixed pulley 122 after being wound around the upper sides thereof move in a second direction D2, which is the opposite direction of the first direction D1. Accordingly, the operation member 140 also moves in a direction K1, which is the same direction as the second direction D2, i.e., move forward, thereby positioning the operation member 140 in an advanced position shown in (b) of FIG. 8.

At this time, the second wire RW may be in a state where no pulling force is applied.

Thereafter, as shown in (b) of FIG. 8, when the first advancing wire FW1 and the second advancing wire FW2 are pulled further in the first direction D1, one region of each of the first advancing wire FW1 and the second advancing wire FW2 is pulled further in the first direction D1, and thus the regions of the first advancing wire FW1 and the second advancing wire FW2 emerging from the lower sides of the first fixed pulley 121 and the second fixed pulley 122 after being wound around the upper sides thereof further move in the second direction D2, which is the opposite direction of the first direction D1. Accordingly, the operation member 140 further moves in the direction K1, which is the same direction as the second direction D2, that is, advances to a position further forward than that shown in (b) of FIG. 8, and as illustrated in (c) of FIG. 8, the operation member 140 advances to a position further forward than that shown in (b) of FIG. 8.

As the operation member 140 moves forward, the second clamp 147 may apply pressure to the lower surface of the first jaw 101, and the first clamp 146 may apply pressure to the upper surface of the second jaw 102.

Accordingly, as the first wire FW is pulled in the first direction D1, the gap between the second jaw 102 and the first jaw 101 may decrease by the operation member 140, and the second jaw 102 may maintain a closed state with respect to the first jaw 101.

Hereinafter, the backward movement of the operation member of the end tool will be described in detail.

Based on FIG. 9, the operation member 140 may move in a rightward direction, i.e., move backward toward the proximal end 100p, and this backward movement is illustrated sequentially in (a), (b), and (c) FIG. 9.

The second wire RW may be connected to one region of the operation member 140 and may be connected, for example, to a rear side of the operation member, specifically, to a region of the blade 142a, which is opposite to the region in which the edge of the blade 142a is formed, among regions of the body 142. The second wire RW may include a plurality of wires, for example, the first retraction wire RW1 and the second retraction wire RW2.

A driving part or a driving transmission part (e.g., a wire, a pulley, or the like) capable of pulling the second wire RW may be connected to the second wire RW, and the second wire RW may be operated according to manual or automatic manipulation. For example, the second wire RW may be pulled by the manipulation part 200 (see FIG. 1).

By pulling the second wire RW, the operation member 140 may move backward.

For example, as shown in (a) of FIG. 9, the second wire RW is pulled in a reverse direction B1 in a state in which the operation member 140 is positioned adjacent to the distal end 100d of the first jaw 101, the operation member 140 connected to the second wire RW moves backward in a direction K2, which is the same direction as the reverse direction B1.

At this point, the first advancing wire FW1 and the second advancing wire FW2 may be in a state in which no pulling force is applied.

When the operation member 140 moves backward (in the direction K2), the regions of the first advancing wire FW1 and the second advancing wire FW2 connected to the operation member 140 move in the first direction D1, which is the same direction as the direction K2, and the regions of the first advancing wire FW1 and the second advancing wire FW2, which are wound around the lower sides of the first fixed pulley 121 and the second fixed pulley 122 and positioned on the upper sides thereof, may move in the second direction D2, which is the opposite direction of the direction K2. Accordingly, the operation member 140 is positioned as shown in (b) of FIG. 9, having moved backward to be closer to the proximal end 100p compared to its position in (a) of FIG. 9.

Thereafter, as shown in (b) of FIG. 9, when the second wire RW is pulled further in the reverse direction B1, the operation member 140 connected to the second wire RW moves backward in the direction K2, which is the same direction as the reverse direction B1. At this point, the first advancing wire FW1 and the second advancing wire FW2 may be in a state in which no pulling force is applied. The regions of the first advancing wire FW1 and the second advancing wire FW2 connected to the operation member 140 move in the first direction D1, which is the same direction as the reverse direction B1, and the regions of the first advancing wire FW1 and the second advancing wire FW2, which are wound around the lower sides of the first fixed pulley 121 and the second fixed pulley 122 and positioned on the upper sides thereof, may move in the second direction D2, which is the opposite direction of the reverse direction B1. Accordingly, the operation member 140 may be positioned as shown in (c) of FIG. 9, having moved backward to be closer to the proximal end 100p compared to its position in (b) of FIG. 9.

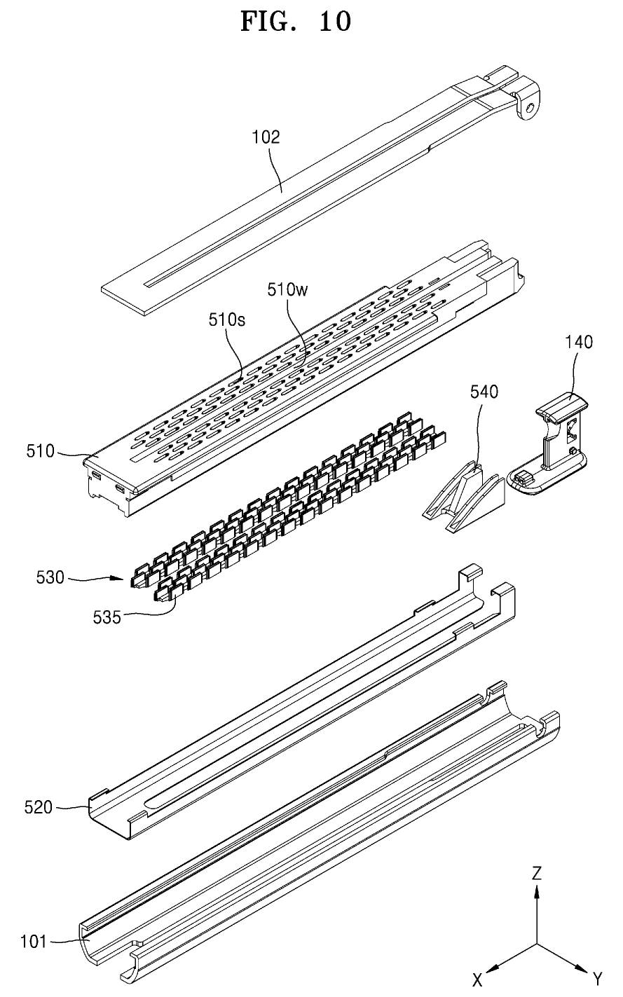

FIG. 10 is an exploded perspective view of the end tool of FIG. 3.

The cartridge 500 may be positioned in the first jaw 101, and for example, the cartridge 500 may be positioned by being coupled to the cartridge accommodation part 101a of the first jaw 101. For example, the cartridge 500 may be integrally formed with the first jaw 101 while the operation member 140 is positioned in the first jaw 101. Further, in an optional embodiment, the cartridge 500 may be configured to be mountable to and dismountable from the first jaw 101.

The cartridge 500 includes the plurality of staples 530 therein to perform suturing of tissue, and performs cutting through the operation member 140. Here, the cartridge 500 may include a cover 510, a housing 520, the staples 530, the withdrawal members 535, and the wedge 540.

The cover 510 may be configured to cover an upper portion of the cartridge accommodation part 101a of the first jaw 101. Staple holes 510s through which the plurality of staples 530 may be ejected to the outside may be formed in the cover 510. As the staples 530, which are accommodated inside the cartridge accommodation part 101a before a stapling operation, are pushed and raised upward by the operation member 140 during a stapling motion, and pass through the staple holes 510s of the cover 510 to be withdrawn out of the cartridge 500, stapling may be performed.

For example, the slit 510w may be defined in the cover 510 along a longitudinal direction of the cover 510. As shown in FIG. 5, the blade 142a of the operation member 140 may protrude to the outside of the cartridge 500 through the slit 510w. As the blade 142a of the operation member 140 passes along the slit 510w, staple-completed tissue may be cut.

The cartridge 500 may include the housing 520, and the staples 530 and the wedge 540 may be positioned in the housing 520 after the housing 520 is positioned in the accommodation part 101a of the first jaw 101, and the cover 510 may be configured to cover an upper portion of the housing 520.

The housing 520 forms an outer shape of the cartridge 500, and may be formed entirely in the form of a hollow box with one surface (upper surface) thereof removed so that the staples 530 and the wedge 540 are accommodated in the hollow box. That is, the housing 520 may be formed in a substantially “U” shape in cross section.

The plurality of staples 530 may be positioned inside the cartridge accommodation part 101a of the first jaw 101. As the operation member 140 linearly moves in one direction, the plurality of staples 530 are sequentially pushed and raised from the inside of the cartridge accommodation part 101a of the first jaw 101 to the outside, thereby performing suturing, that is, stapling. Here, the staples 530 may include a material that is durable and does not have an abnormal effect on the human body, such as titanium, stainless steel, or the like.

For example, the withdrawal members 535 may be further disposed between the cartridge accommodation part 101a of the first jaw 101 and the staples 530. For example, it may be said that the staple 530 is disposed above the withdrawal member 535. In this case, the operation member 140 is linearly moved in one direction to push the withdrawal member 535 upward, and the withdrawal member 535 may push the staple 530 upward.

As such, the operation member 140 may be described as pushing the staples 530 upward in both the case in which the operation member 140 directly pushes the staples 530 upward and the case in which the operation member 140 pushes the withdrawal members 535 upward and the withdrawal members 535 pushes the staples 530 upward (i.e., the operation member 140 indirectly pushes the staples 530 upward).

As described above, the operation member 140 may be positioned inside the cartridge accommodation part 101a of the first jaw 101. Further, the operation member 140 may include the wedge 540 or may be used together with the wedge 540, and when the operation member 140 moves, the wedge 540 may move together with the operation member 140, and as a result, the wedge 540 may directly push the staples 530 upward or push the withdrawal members 535 upward, which in turn pushes the staples 530 upward.

As described above, as the first wire FW moves, i.e., the first wire FW is pulled, the operation member 140 connected thereto may move forward toward the distal end 100d of the first jaw 101.

The forward movement of the operation member 140 may cause the wedge 540 to push the withdrawal member 535 upward, which may also cause the staple 530 to rise, and at the same time, cutting using the blade 142a of the operation member 140 may be performed. In some embodiments, in the case of the end tool 100 in which the second wire RW is connected to the operation member 140, the second wire RW may be pulled to cause the operation member 140 to move backward toward the proximal end 100p of the first jaw 101.

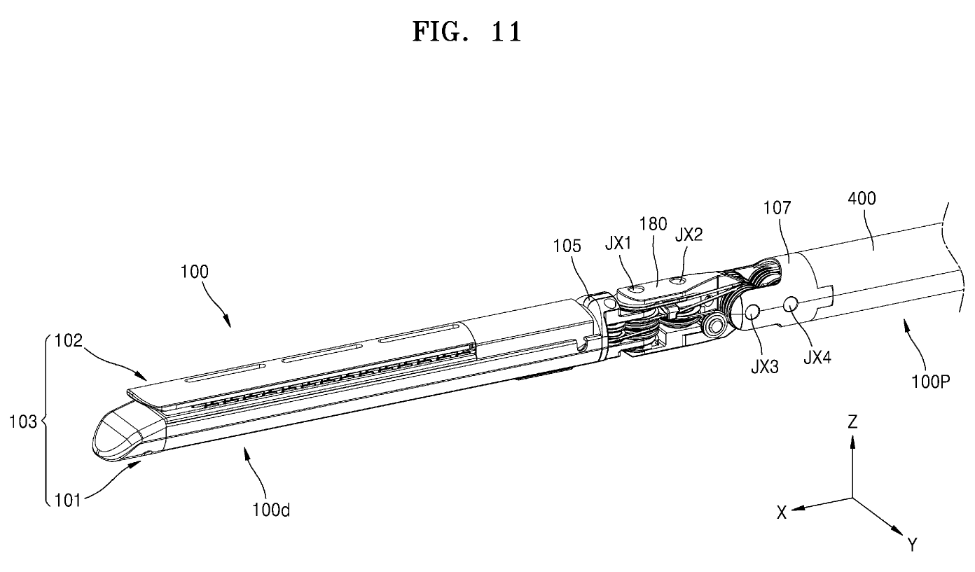

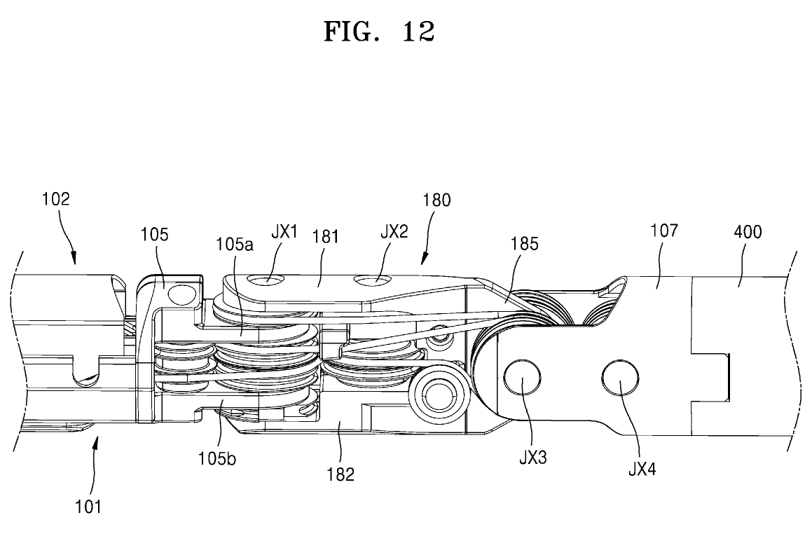

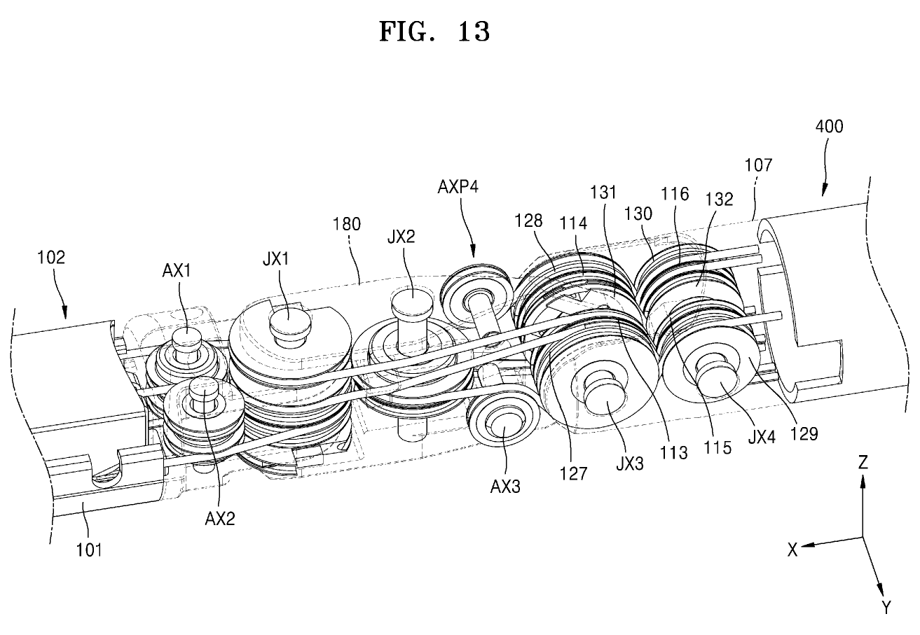

FIG. 11 is a perspective view illustrating the end tool according to an embodiment of the present disclosure. FIG. 12 is a perspective view for describing an end tool hub and a pitch hub of the end tool of FIG. 11. FIG. 13 is a perspective view for describing a rotational shaft and pulley structure of the end tool of FIG. 11.

Referring to FIGS. 11 to 13, the end tool 100 of the surgical instrument of the present embodiment may include one or more members, such as joint members, that connect the jaw 103 to the connection part 400. In an optional embodiment, the end tool 100 may include an end tool hub 180 and a pitch hub 107.

The end tool hub 180 may be disposed to connect the jaw 103 to the connection part 400. For example, the end tool hub 180 may be a member connecting the connection part 400 to the first jaw 101 and the second jaw 102. Alternatively, the end tool hub 180 may be disposed to connect the jaw 103 to the pitch hub 107. That is, the end tool hub 180 may be a connection member interposed between the jaw 103 and the pitch hub 107.

The end tool hub 180 may be configured to internally accommodate at least a portion of the first jaw 101.

In some embodiments, the pitch hub 107 may be configured to be axially coupled to the end tool hub 180 and to be rotatable relative to the end tool hub 180. For example, the end tool hub 180 and the pitch hub 107 may be axially coupled to each other through the third rotational shaft JX3. Specific details of the pitch hub 107 will be described later.

The end tool hub 180 may include a pair of jaw pulley coupling parts 181 and 182 and a pitch pulley coupling part 185.

In detail, the pair of jaw pulley coupling parts 181 and 182 are formed to face each other so that a plurality of pulleys can be accommodated therein. In some embodiments, a through hole is formed in each of the jaw pulley coupling parts 181 and 182 so that the first rotational shaft JX1 passes therethrough and axially couple the jaw pulley coupling parts 181 and 182 to the pulleys.

The pair of jaw pulley coupling parts 181 and 182 may be connected to each other by the pitch pulley coupling part 185. That is, the pair of jaw pulley coupling parts 181 and 182 are coupled to each other by the pitch pulley coupling part 185 formed in a direction substantially perpendicular thereto, so that the pair of jaw pulley coupling parts 181 and 182 and the pitch pulley coupling part 185 form a substantially “C” shape, in which a plurality of pulleys are accommodated.

In other words, it may be expressed that the pair of jaw pulley coupling parts 181 and 182 are formed to extend in an X-axis direction from both end portions of the pitch pulley coupling part 185 formed to be elongated in a Z-axis direction.

For example, a pitch pulley 131 around which a pitch wire may be wound may be coupled to the pitch pulley coupling part 185. Alternatively, the pitch pulley 131 is not a member that is rotated around a predetermined axis like the original meaning pulley does, and it may be said that the pitch pulley 131 is formed to be fixed as a portion of the end tool hub 180 and performs some similar functions of a pulley by winding a wire therearound. Here, the pitch pulley coupling part 185 may be formed on an XZ plane. In some embodiments, a through hole through which the third rotational shaft JX3 may be inserted may be formed in the pitch pulley coupling part 185.

The first rotational shaft JX1 and a second rotational shaft JX2 may be inserted through the end tool hub 180, and the end tool hub 180 may internally accommodate at least some of pulleys axially coupled to the first rotational shaft JX1. In addition, the end tool hub 180 may internally accommodate at least some of pulleys axially coupled to the second rotational shaft JX2.

In an embodiment, the second rotational shaft JX2 may be disposed adjacent and parallel to the first rotational shaft JX1.

In another embodiment, the second rotational shaft JX2 may be disposed alongside the first rotational shaft JX1 but may not be parallel to the first rotational shaft JX1. That is, the first rotational shaft JX1 is disposed in the Z-axis direction in the drawing, whereas the second rotational shaft JX2 may be disposed obliquely at a predetermined angle with respect to a Z-axis.

In some embodiments, the pitch pulley 131 serving as an end tool pitch pulley may be formed at one end portion of the end tool hub 180. The pitch pulley 131 may be integrally formed with the end tool hub 180 as one body. That is, a disk-shaped pulley is formed at one end portion of the end tool hub 180, and a groove around which a wire may be wound may be formed on an outer circumferential surface of the pulley. Alternatively, the pitch pulley 131 may be formed as a separate member from the end tool hub 180 to be coupled to the end tool hub 180. The pitch wire is coupled to the pitch pulley 131 serving as an end tool pitch pulley, and when the pitch pulley 131 rotates around the third rotational shaft JX3, the end tool hub 180 also rotates together with the pitch pulley 131, thereby performing a pitch motion.

In some embodiments, the pitch hub 107 may be configured to be axially coupled to the end tool hub 180 and to be rotatable relative to the end tool hub 180. For example, the end tool hub 180 and the pitch hub 107 may be axially coupled to each other through the third rotational shaft JX3.

The pitch hub 107 may have a disk-shaped body formed on one side thereof and coupled to the connection part 400, with an extension portion extending from the body toward the end tool hub 180.

For example, the third rotational shaft JX3 and a fourth rotational shaft JX4, which will be described later, may be inserted through the extension portion extending toward the end tool hub 180, and the pitch hub 107 and the end tool hub 180 (and the pitch pulley 131) may be axially coupled to each other by the third rotational shaft JX3. Thus, the end tool hub 180 and the pitch pulley 131 may be formed to be rotatable around the third rotational shaft JX3 with respect to the pitch hub 107.

Further, the pitch hub 107 may internally accommodate at least some of pulleys axially coupled to the third rotational shaft JX3. In addition, the pitch hub 107 may internally accommodate at least some of pulleys axially coupled to the fourth rotational shaft JX4.

Here, the third rotational shaft JX3 may function as a pitch rotational shaft, and the fourth rotational shaft JX4 may function as an auxiliary pulley rotational shaft.

That is, the auxiliary pulley rotational shaft JX4 may be disposed parallel to and adjacent to the pitch rotational shaft JX3.

In some embodiments, the pitch hub 107 may further include a pitch auxiliary pulley 132 that rotates around the fourth rotational shaft JX4.

Referring to FIGS. 11 to 13, in the end tool according to an embodiment of the present disclosure, a plurality of rotational shafts may be disposed in the end tool hub 180 and the pitch hub 107.

In an embodiment, in the end tool hub 180, the first rotational shaft JX1 may be positioned adjacent to the first jaw 101, and the second rotational shaft JX2, which is parallel to the first rotational shaft JX1, may be positioned adjacent to the first rotational shaft JX1. That is, the first rotational shaft JX1 and the second rotational shaft JX2 may be sequentially disposed from the proximal end 100p toward the distal end 100d.

For example, in the pitch hub 107, the third rotational shaft JX3 axially coupled to the end tool hub 180 may be disposed, and the fourth rotational shaft JX4 that is parallel to the third rotational shaft JX3 may be positioned adjacent to the third rotational shaft JX3.

Here, pulleys may be axially coupled to the first to fourth rotational shafts JX1 to JX4. In some embodiments, a first jaw pulley 111 and a second jaw pulley 112 may be axially coupled to the first rotational shaft JX1. This will be described in detail later.

In some embodiments, pulleys may be axially coupled to the first rotational shaft JX1. For example, a pulley 123 adjacent to the first jaw pulley 111 and a pulley 124 adjacent to the second jaw pulley 112 may be disposed. That is, the first jaw pulley 111, the pulley 123, the pulley 124, and the second jaw pulley 112 may be axially coupled to the same rotational shaft.

That is, the pulley is disposed such that at least a portion of the wire unit is in contact therewith, thereby guiding a traveling path of the wire unit. This will be described in detail later.

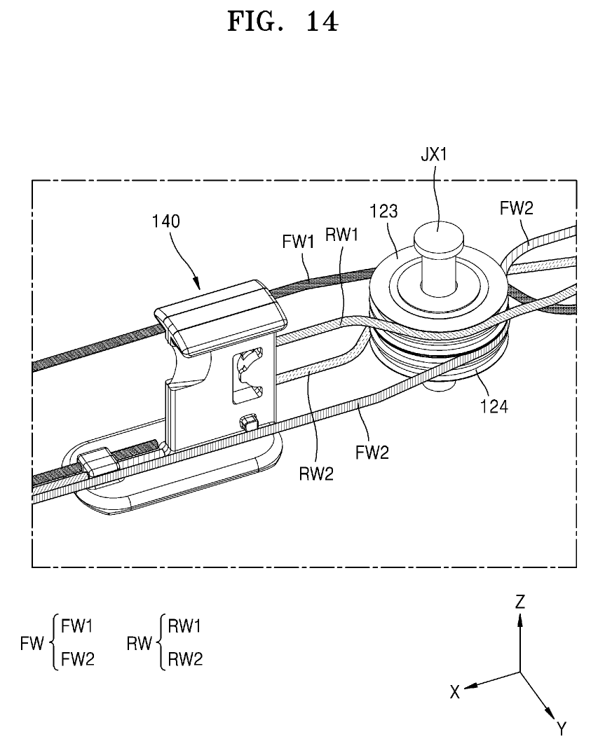

FIG. 14 is a perspective view for describing a connection relationship among the pulleys, the operation member, and the wire units of FIG. 11.

The wire units may include the first wire FW and the second wire RW. In this case, the first wire FW may include the first advancing wire FW1 and the second advancing wire FW2, and the second wire RW may include the first retraction wire RW1 and the second retraction wire RW2.

Referring to FIG. 14, the first advancing wire FW1 and the first retraction wire RW1 may be wound around the pulley 123, and the second advancing wire FW2 and the second retraction wire RW2 may be wound around the pulley 124.

Here, the first advancing wire FW1 and the first retraction wire RW1 may form a pair of wires, and the second advancing wire FW2 and the second retraction wire RW2 may form a pair of wires.

In an embodiment, with respect to the first rotational shaft JX1 of the pulley 123, the first advancing wire FW1 may be positioned on one side of the pulley 123, and the first retraction wire RW1 may be positioned on another side of the pulley 123.

For example, a pair of wires FW1 and RW1 wound around the pulley 123 may be positioned in opposite directions with respect to the first rotational shaft JX1.

In some embodiments, when the pulley 123 rotates in one direction, the first advancing wire FW1 and the first retraction wire RW1 wound around the pulley 123 may move in different directions.

In some embodiments, when the pulley 123 rotates counterclockwise, the first advancing wire FW1 in contact with the pulley 123 may move in a direction in which the operation member moves forward, and the first retraction wire RW1 may move in a direction in which the operation member moves backward.

For example, when the first advancing wire FW1 and the first retraction wire RW1 move, the movements of the two wires may cause the pulley 123 to rotate in the same direction.

In an embodiment, the pulley 123 and the pulley 124 may be axially coupled to one first rotational shaft JX1. In some embodiments, based on the first rotational shaft JX1, the second advancing wire FW2 may be positioned on one side of the first rotational shaft JX1, and the second retraction wire RW2 may be positioned on another side of the first rotational shaft JX1.

In some embodiments, when the pulley 124 rotates in one direction, the second advancing wire FW2 and the second retraction wire RW2 wound around the pulley 124 may move in different directions.

The first advancing wire FW1 and the second retraction wire RW2 may be positioned on one side of the rotational shaft, and the first retraction wire RW1 and the second advancing wire FW2 may be positioned on another side of the rotational shaft.

For example, the first advancing wire FW1 and the second advancing wire FW2 may be positioned on different sides of the rotational shaft. In some embodiments, the first retraction wire RW1 and the second retraction wire RW2 may be positioned on different sides of the rotational shaft.

Referring to FIGS. 5, 8, 9, and 14, a pair of wires may be wound around each of the pulleys 123 and 124, and each pair of wires may be configured to control one degree of freedom of the jaw 103.

In some embodiments, the first jaw 101 may be configured to accommodate at least one region of the operation member 140 that is movable in at least one direction. In some embodiments, the first wire FW and the second wire RW may be connected to the operation member so that the operation member can move in at least one direction. The first wire FW and the second wire RW may control the degrees of freedom of the operation member.

For example, among the pair of wires, the first advancing wire FW1 may be an advancing wire that moves the operation member toward the distal end 100d of the end tool, and the first retraction wire RW1 may be a retraction wire that moves the operation member toward the proximal end 100p of the end tool.

That is, the first advancing wire FW1 and the first retraction wire RW1 may be wires that control the degrees of freedom related to the forward or backward movement of the operation member.

In some embodiments, the first advancing wire FW1 and the first retraction wire RW1 may move in different directions and may move the operation member forward or backward. Accordingly, the first advancing wire FW1 and the first retraction wire RW1 may move in opposite directions at a 1:1 ratio.

As the first advancing wire FW1 and the first retraction wire RW1 move in opposite directions at a 1:1 ratio as described above, the first advancing wire FW1 and the first retraction wire RW1 may be positioned on opposite sides of the rotational shaft when wound around the pulley 123.

By positioning first advancing wire FW1 and the first retraction wire RW1 as described above, the pulley 123 may rotate in one direction when the first advancing wire FW1 and the first retraction wire RW1 move.

For example, a direction in which the pulley 123 is rotated by the first advancing wire FW1 and a direction in which the pulley 123 is rotated by the first retraction wire RW1 may be the same.

Similarly, the second advancing wire FW2 and the second retraction wire RW2 may be connected to the operation member so that the operation member can move in at least one direction. The second advancing wire FW2 and the second retraction wire RW2 may control the degrees of freedom of the operation member.

For example, the second advancing wire FW2 may be an advancing wire that moves the operation member toward the distal end 100d of the end tool, and the second retraction wire RW2 may be a retraction wire that moves the operation member toward the proximal end 100p of the end tool.

That is, the second advancing wire FW2 and the second retraction wire RW2 may be wires that control the degrees of freedom related to the forward or backward movement of the operation member.

In some embodiments, the second advancing wire FW2 and the second retraction wire RW2 move in different directions and may move the operation member forward or backward. Accordingly, the second advancing wire FW2 and the second retraction wire RW2 may move in opposite directions at a 1:1 ratio.

As the second advancing wire FW2 and the second retraction wire RW2 move in opposite directions at a 1:1 ratio as described above, when the second advancing wire FW2 and the second retraction wire RW2 are wound around the pulley 124, the second advancing wire FW2 and the second retraction wire RW2 may be positioned on opposite sides of the rotational shaft.

By positioning the second advancing wire FW2 and the second retraction wire RW2 as described above, when the second advancing wire FW2 and the second retraction wire RW2 move, the pulley 124 may rotate in one direction.

For example, a direction in which the pulley 124 is rotated by the second advancing wire FW2 and a direction in which the pulley 124 is rotated by the second retraction wire RW2 may be the same.

In some embodiments, the first wire FW and the second wire RW may be different wires. For example, the first advancing wire FW1 and the first retraction wire RW1 forming a pair may be different wires from each other, and the second advancing wire FW2 and the second retraction wire RW2 forming another pair may also be different wires from each other.

The first wire FW and the second wire RW may be formed of different materials. In some embodiments, the first wire FW and the second wire RW may be formed of materials having different strengths.

For example, the first wire FW may be a wire including a metal material, and the second wire RW may be a wire including a polymer.

Alternatively, the first wire FW may include tungsten, and the second wire RW may include stainless steel.

Alternatively, the first wire FW may be a wire in which one of the constituent elements accounts for 95% or more by weight, and the second wire RW may be a wire in which none of the constituent elements accounts for 80% or more by weight. For example, the first wire FW may include a pure metal, and the second wire RW may include an alloy.

Alternatively, the first wire FW may be described as including tungsten having a purity of 95% or more, and the second wire RW may be described as including stainless steel having an iron content of less than 80%.

The first wire FW and the second wire RW being different from each other may indicate that they have different diameters or tensile strengths.

For example, the first wire FW may have a larger diameter than the second wire RW. In an optional embodiment, when the first wire FW has a diameter of 0.5 mm to 0.6 mm, the second wire RW may have a diameter of 0.4 mm to 0.5 mm.

Alternatively, the first wire FW may have a greater tensile strength than the second wire RW. For example, when the same load is applied, the first wire FW may be elongated relatively less than the second wire RW.

Alternatively, the first wire FW may have a greater tensile strength than the second wire RW. For example, the maximum stress that the first wire FW can withstand may be greater than the maximum stress that the second wire RW can withstand.

Alternatively, a yield point of the first wire FW may be positioned at a point corresponding to 80% or more of a tensile strength of the first wire FW, and a yield point of the second wire RW may be positioned at a point corresponding to 80% or less of a tensile strength of the second wire RW.

Here, the yield point refers to the point at which a material transitions from elastic deformation to plastic deformation, i.e., the point at which the material undergoes permanent deformation and does not return to its original shape when the external load is removed.

As described above, the wire for controlling one degree of freedom may generally form a closed loop. Even in the wire unit according to an embodiment of the present disclosure, when the pulley connected to a rotational shaft rotates in one direction, the first wire FW and the second wire RW connected to the pulley may move in opposite directions.

In this case, a ratio at which the wires move in opposite directions may be different from 1:1. For example, when the pulley rotates in one direction and an angle formed between the wires and a central axis of rotation of the pulley changes, a ratio of unwinding and winding of the pair of wires may become different from a 1:1 ratio.

In some embodiments, referring to FIG. 14, when the pulley 123 rotates clockwise, a length of the first advancing wire FW1 moving toward the distal end of the jaw and a length of the first retraction wire RW1 moving toward the proximal end of the jaw may become different from a 1:1 ratio.

When the length of the first advancing wire FW1 moving toward the distal end 100d of the jaw 103 is greater than the length of the first retraction wire RW1 moving toward the proximal end 100p of the jaw 103, a total length of a loop formed by the pair of wires may decrease, which may excessively increase the tension of the wires and cause damage to the wires.

In the opposite case, when the length of the first advancing wire FW1 moving toward the distal end 100d of the jaw 103 is less than the length of the first retraction wire RW1 moving toward the proximal end 100p of the jaw 103, the total length of the loop formed by the pair of wires may increase, which may excessively reduce the initial tension of the wires and cause the wires to deviate from the path.

When one of the first wire FW and the second wire RW is formed of a material that is relatively more stretchable than the other, the effect of a change in the total length of the loop formed by the pair of wires on the overall wire tension may be relatively reduced.

For example, when one of the first wire FW and the second wire RW is formed of a material that is relatively more stretchable than the other, even when the total length of the loop formed by the pair of wires connected to one pulley decreases, the overall tension applied to the wires may not increase excessively.

In some embodiments, even when the total length of the loop formed by the pair of wires increases, the overall tension applied to the wires may not decrease excessively.

In the end tool according to an embodiment of the present disclosure, the first wire FW and the second wire RW, which control one degree of freedom of the jaw 103, are formed of different materials. As a result, even when the length of the loop formed by the pair of wires increases or decreases, the tension can be stably maintained within a preset range.

In addition, since the first wire FW, which transmits a driving force to move the operation member 140 forward, is formed of a material having relatively higher rigidity than the second wire RW, which transmits a driving force to move the operation member 140 backward, the first wire FW can deliver sufficient force required for driving the end tool 100 while maintaining its performance even after prolonged and repeated use.

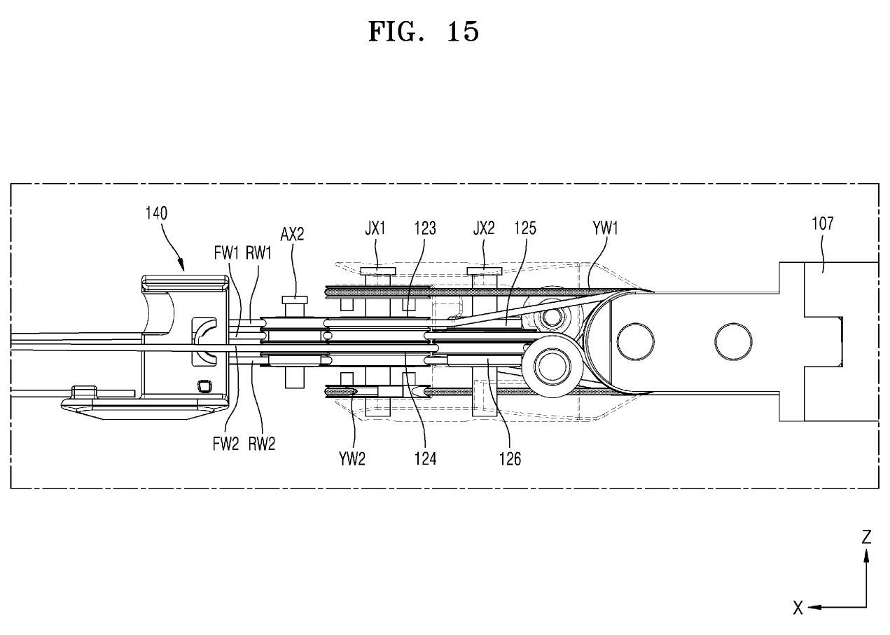

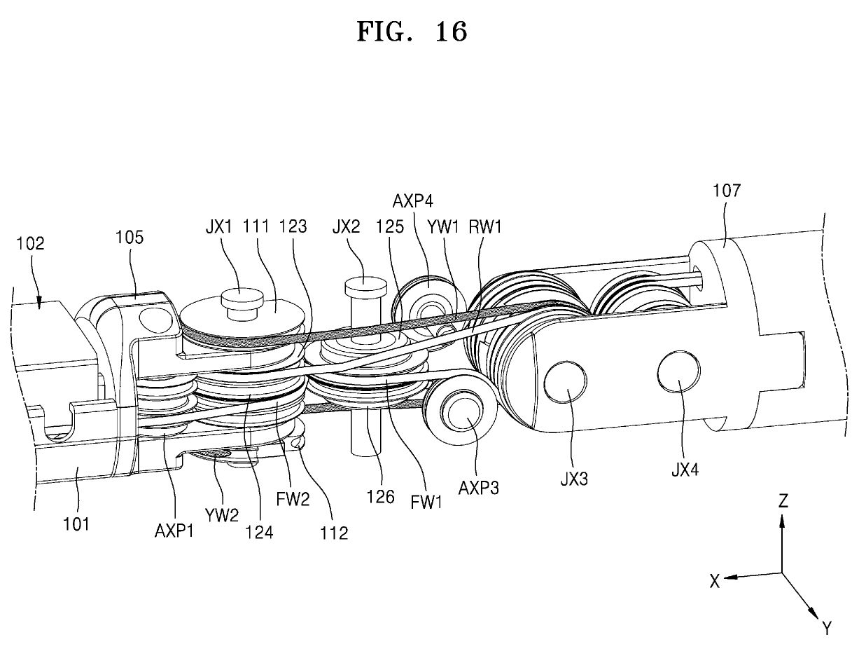

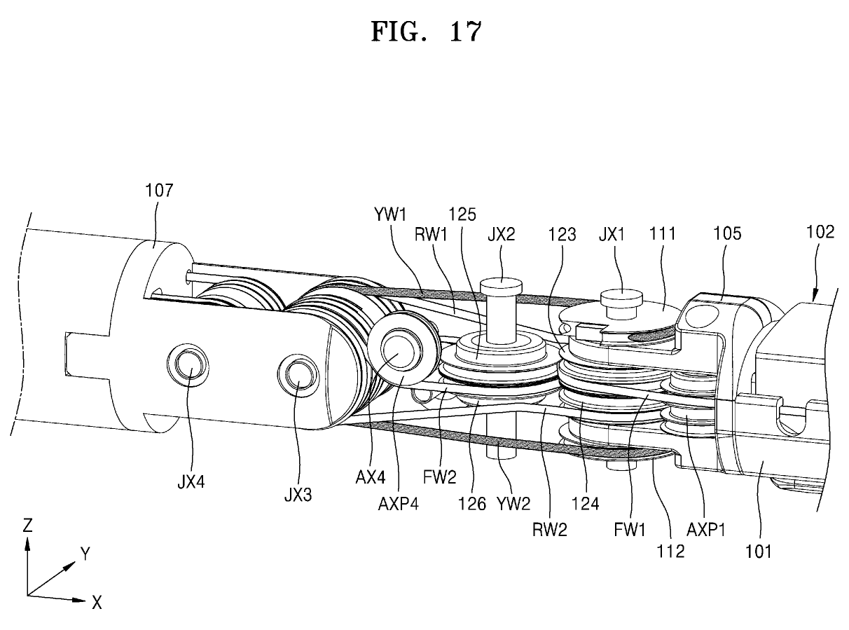

FIG. 15 is a side view of the end tool of FIG. 11 with the end tool hub removed, FIG. 16 is a perspective view of the end tool of FIG. 11 with the end tool hub removed, and FIG. 17 is a perspective view of the end tool of FIG. 16 viewed from another direction.

As described above, the end tool 100 may be connected to the connection part 400, and the end tool 100 may rotationally move around the first shaft and also rotationally move around a second shaft with respect to the connection part 400.

For example, the end tool 100 may perform a pitch motion, i.e., a vertical rotational motion (rotational motion in the Z-axis direction) based on FIGS. 1 and 2, and the end tool 100 may perform a yaw motion, i.e., a horizontal rotational motion (rotational motion in a direction along an x,y-plane) based on FIGS. 1 and 2. A rotation axis of the pitch motion and a rotation axis of the yaw motion may be positioned in directions that may intersect or be perpendicular to each other.

The end tool 100 may further include a third wire YW that transmits a driving force for rotational motion around one axis, and the pulleys 111 and 112 around which the third wire YW may be wound.

The third wire YW may be a pitch wire that transmits a driving force to cause the end tool 100 to perform a pitch motion. The pitch wire may include two or more wires.

In some embodiments, the third wire YW may be a yaw wire that transmits a driving force to cause the end tool 100 to perform a yaw motion. The third wire YW may include two or more wires, for example, a first yaw wire YW1 and a second yaw wire YW2.

Hereinafter, in the present specification, for convenience of description, the third wire YW will be described as a plurality of yaw wires YW1 and YW2.

Referring to FIGS. 11 and 15 to 17, the first rotational shaft JX1 may connect the end tool hub 180 to the first jaw 101. In some embodiments, the first jaw 101 may include a jaw connection part 105, and the jaw connection part 105 may include a first shaft coupling part 105a and a second shaft coupling part 105b, which extend toward the end tool hub 180.

Here, the first rotational shaft JX1 may pass through the jaw pulley coupling parts 181 and 182 of the end tool hub 180, the first shaft coupling part 105a, and the second shaft coupling part 105b to axially couple the end tool hub 180 to the jaw connection part 105. For example, the first rotational shaft JX1 may function as a yaw rotational shaft. In other words, the end tool hub 180 and the jaw connection part 105 may relatively rotate around the first rotational shaft JX1.

The first jaw pulley 111 may be positioned between the jaw pulley coupling part 181 at the upper side and the first shaft coupling part 105a, and the second jaw pulley 112 may be positioned between the jaw pulley coupling part 182 at the lower side and the second shaft coupling part 105b.

The first yaw wire YW1 and the second yaw wire YW2 may be wound around the first jaw pulley and the second jaw pulley, respectively.

As the first yaw wire YW1 is pulled toward the proximal end 100p, the first jaw pulley 111 is rotated, and a rotational force may be transmitted to the first shaft coupling part 105a coupled to the first jaw pulley 111. Likewise, when the second yaw wire YW2 is pulled toward the proximal end 100p, the second jaw pulley 112 is rotated, and a rotational force may be transmitted to the second shaft coupling part 105b coupled to the second jaw pulley 112.

For example, the pulley 123 and the pulley 124 may be positioned between the first shaft coupling part 105a and the second shaft coupling part 105b. That is, the first rotational shaft JX1 may pass through the pulley 123 and the pulley 124 and be axially coupled to the first shaft coupling part 105a and the second shaft coupling part 105b.

In other words, the pulley 123 and the pulley 124 may be axially coupled to the same rotational shaft as the first jaw pulley 111 and the second jaw pulley 112. For example, the pulley 123 and the pulley 124 may be axially coupled to the yaw rotational shaft.

Referring to FIGS. 13 and 15 to 17, the first yaw wire YW1 may sequentially come into contact with the first jaw pulley 111, a pulley 113, and a pulley 115 while extending from the distal end 100d toward the proximal end 100p of the end tool.

The second yaw wire YW2 may sequentially come into contact with the second jaw pulley 112, a pulley 114, and a pulley 116 while moving from the distal end 100d toward the proximal end 100p of the end tool.

In some embodiments, the pulley 131 may function as a pitch pulley, and pulley 132 may function as a pitch auxiliary pulley.

In some embodiments, pulleys may also be disposed on the pitch hub 107. For example, a pulley 127 and a pulley 128 may be axially coupled to the third rotational shaft JX3, and a pulley 129 and a pulley 130 may be axially coupled to the fourth rotational shaft JX4.

In some embodiments, based on an imaginary plane passing through the first rotational shaft JX1 and the second rotational shaft JX2, the pulley 127 may be positioned on one side, and the pulley 128 may be positioned on another side. For example, the pulley 127 may be positioned on one side of the pitch hub 107 and the pulley 128 may be placed on another side of the pitch hub 107.

Here, the third rotational shaft JX3 may pass through the pitch pulley coupling part 185 of the end tool hub 180 to axially couple and connect the end tool hub 180 to the pitch hub 107. That is, the third rotational shaft JX3 may function as a pitch rotational shaft. For example, the pulleys 127 and 128 may be axially coupled to the pitch rotational shaft, and the pitch wire may be wound around the pulleys 127 and 128.

In some embodiments, the wire unit and the third wire YW may be different wires. For example, the first wire FW, which transmits a driving force to move the operation member 140 forward toward the distal end of the jaw, and the second wire RW, which transmits a driving force to move the operation member 140 backward toward the proximal end of the jaw may be wires different from the pitch wires.

In addition, the first wire FW, which transmits a driving force to move the operation member 140 forward toward the distal end of the jaw, and the second wire RW, which transmits a driving force to move the operation member 140 backward toward the proximal end of the jaw, may be wires different from the yaw wires.

Hereinafter, the description is provided based on the assumption that the first wire FW and the third wire YW are different wires.

The first wire FW and the third wire YW may be formed of different materials. In some embodiments, the first wire FW and the third wire YW may be formed of materials having different strengths.

For example, the first wire FW may be a wire including a metal material, and the third wire YW may be a wire including a polymer.

Alternatively, the first wire FW may include tungsten and the third wire YW may include stainless steel.

Alternatively, the first wire FW may be a wire in which one of the constituent elements accounts for 95% or more by weight, and the third wire YW may be a wire in which none of the constituent elements accounts for 80% or more by weight. For example, the first wire FW may include a pure metal, and the third wire YW may include an alloy.

Alternatively, the first wire FW may be described as including tungsten having a purity of 95% or more, and the third wire YW may be described as including stainless steel having an iron content of less than 80%.

The first wire FW and the third wire YW being different wires may indicate that they have different diameters or tensile strengths.

For example, the first wire FW may have a larger diameter than the third wire YW. In an optional embodiment, when the first wire FW has a diameter of 0.5 mm to 0.6 mm, the third wire YW may have a diameter of 0.4 mm to 0.5 mm.

Alternatively, the first wire FW may have a greater tensile strength than the third wire YW. For example, under the same loading conditions, the first wire FW may be elongated relatively less than the third wire YW.

Alternatively, the first wire FW may have a greater tensile strength than the third wire YW. For example, the maximum stress that the first wire FW can withstand may be greater than the maximum stress that the third wire YW can withstand.

Alternatively, a yield point of the first wire FW may be positioned at a point corresponding to 80% or more of a tensile strength of the first wire FW, and a yield point of the third wire YW may be positioned at a point corresponding to 80% or less of a tensile strength of the third wire YW.

In general, when a material with relatively high tensile strength is used for a wire, there have been issues such as high unit cost, difficulty in procurement, and complexity in processing, such as cutting the wire.

In the end tool 100 according to an embodiment of the present disclosure, a material with high tensile strength is used for the first wire FW, to which a relatively large force is applied, and a material with relatively low tensile strength is used for the third wire YW, which transmits a driving force for simple rotational motion of the end tool 100. Accordingly, production costs can be reduced, and process efficiency in the manufacturing process of the surgical instrument can be improved.

According to an embodiment of the present disclosure, an end tool of a surgical instrument used in laparoscopic surgery or various other surgical procedures can be driven by different types of wires, so that a tension acting on a pair of wires that control one degree of freedom can be maintained within a preset range.

The present disclosure has been described above with a focus on exemplary embodiments. It will be understood by those skilled in the art that various changes in form and details may be made therein without departing from the essential features of the present disclosure. Therefore, the disclosed embodiments should be considered in descriptive sense only and not for purposes of limitation. The scope of the present disclosure is defined not by the detailed description of the disclosure but by the appended claims, and all differences within the scope will be construed as being included in the present disclosure.

What is claimed is:

1. An end tool of a surgical instrument, the end tool comprising:
a jaw configured to accommodate at least one region of an operation member configured to be movable in at least one direction; and
at least one wire including a first wire and a second wire, which are both connected to the operation member to allow the operation member to move in the at least one direction,
wherein the first wire and the second wire are different wires.

2. The end tool of claim 1, wherein
the first wire is configured to transmit a driving force to cause the operation member to move forward toward a distal end of the jaw, and
the second wire is configured to transmit a driving force to cause the operation member to move backward toward a proximal end of the jaw.

3. The end tool of claim 2, wherein the first wire and the second wire include different materials.

4. The end tool of claim 3, wherein
the first wire includes a metal material, and
the second wire includes a polymer.

5. The end tool of claim 2, wherein
one of the first wire and the second wire includes stainless steel, and another of the first wire and the second wire includes tungsten.

6. The end tool of claim 5, wherein
the first wire includes the tungsten having a purity of 95% or more, and
the second wire includes the stainless steel having an iron content of less than 80%.

7. The end tool of claim 2, wherein the first wire has a greater tensile strength than the second wire.

8. The end tool of claim 1, further comprising at least one pulley having a predetermined rotational central axis and configured to contact the at least one wire,
wherein the first wire and the second wire are wound around the at least one pulley to control at least one degree of freedom of the jaw.

9. The end tool of claim 8, wherein, when the at least one pulley rotates in one direction, the first wire and the second wire move in different directions.

10. An end tool of a surgical instrument, the end tool comprising:
a jaw configured to accommodate at least one region of an operation member configured to be movable in at least one direction;
a first wire connected to the operation member to allow the operation member to move in the at least one direction; and
a third wire configured to transmit a driving force to cause the jaw to perform a rotational motion around a first shaft,
wherein the first wire and the third wire are different wires.

11. The end tool of claim 10, wherein
the first wire is configured to transmit a driving force to cause the operation member to move forward toward a distal end of the jaw, and
the third wire is configured to correspond to a yaw motion of the end tool.

12. The end tool of claim 10, wherein
the first wire is configured to transmit a driving force to cause the operation member to move forward toward a distal end of the jaw, and
the third wire is configured to correspond to a pitch motion of the end tool.

13. The end tool of claim 11, wherein the first wire and the third wire include different material.

14. The end tool of claim 13, wherein
the first wire includes a metal material, and
the third wire includes a polymer.

15. The end tool of claim 11, wherein
the first wire includes tungsten having a purity of 95% or more, and
the third wire includes stainless steel having an iron content of less than 80%.

16. The end tool of claim 11, wherein the first wire has a greater tensile strength than the third wire.

17. A surgical instrument comprising:
the end tool of claim 1;
a manipulation part configured to control a motion of the end tool; and
a connection part configured to connect the manipulation part to the end tool.

18. The surgical instrument of claim 17, wherein the end tool is configured to perform a yaw rotation around a first shaft and a pitch rotation around a second shaft different from the first shaft.

19. A pulley fixing pin applicable to a surgical instrument, the pulley fixing pin comprising:
a body portion extending in a longitudinal direction and configured to pass through a fixed pulley around which a wire is wound; and
a flange portion disposed on one side of the body portion,
wherein a first diameter of an outermost circumferential surface of the flange portion is greater than a second diameter of an outer circumferential surface of the body portion.

20. The pulley fixing pin of claim 19, wherein the first diameter of the outer circumferential surface of the flange portion increases in a direction away from the body portion.
